(19) United States
(12) Patent Application Publication
(10) Pub. No.: US 2009/0280064 A1
(43) Pub. Date: Nov. 12, 2009

(54) TRANSDERMAL DELIVERY OF OPTICAL, SPECT, MULTIMODAL, DRUG OR BIOLOGICAL CARGO LADEN NANOPARTICLE(S) IN SMALL ANIMALS OR HUMANS

(76) Inventors: Rao Papineni, Brandord, CT (US); Douglas Lincoln Vizard, Durham, CT (US); William E. McLaughlin, Guilford, CT (US); John William Harder, Rochester, NY (US); David L. Patton, Lebannon, PA (US); Guizhi Li, Rochester, NY (US)

Correspondence Address: Susan L. Parulski;Patent Legal Staff, Carestream Health, Inc., 150 Verona Street, Rochester, NY, 14608, US

(21) Appl. No.: 12/202,681

(22) Filed: Sep. 2, 2008

Related U.S. Application Data
(63) Continuation-in-part of application No. 11/165,849, filed on Jun. 24, 2005.
Application No. 11/165,849 is a continuation-in-part of application No. 11/401,343, filed on Apr. 10, 2006.
Application No. 11/401,343 is a continuation-in-part of application No. 12/221,839, filed on Aug. 7, 2008.

(51) Int. Cl. A61K 49/06; A61K 49/00; A61K 49/04

(52) U.S. Cl. 424/9.3; 424/9.1; 424/9.4; 424/9.6

(57) ABSTRACT

A method and a device are disclosed for transdermal delivery to an animal or human of biological cargo-laden nanoparticles. The particles may include multimodal optical molecular imaging probes. The particles may be delivered by providing them in a form that can be absorbed through the skin and applying them to the skin of an animal or human. The application may be accomplished using biological cargo-laden nanoparticles in a device attachable to the skin. The device may be attached directly to the skin by a device containing a vasodilating agent or agents, or micro needles, or multi-layer time release material. The biological cargo-laden nanoparticles may comprise drugs, vaccines, bio-pharmaceuticals, imaging contrast agents, multimodal imaging contrast agents, biomolecules, or anti-infectives. The device may include a first plurality of different types of biological cargo-laden nanoparticles located in a corresponding second plurality of separate time release layers.

### CROSS REFERENCE TO RELATED APPLICATIONS

This application is a continuation-in-part of the following commonly assigned, copending U.S. patent applications, the priority of each of which is claimed and each of which is incorporated by reference:

regular Ser. No. 11/165,849 filed on Jun. 24, 2005 by Bringley et al. entitled “NANOPARTICLE BASED SUBSRATE FOR IMAGE CONTRAST AGENT FABRICATION”;

regular Ser. No. 11/401,343 filed on Apr. 10, 2006 by Leon et al. entitled “NANOGEL-BASED CONTRAST AGENTS FOR OPTICAL MOLECULAR IMAGING”; and

regular Ser. No. 12/221,839 filed on Aug. 7, 2008 by Li et al entitled “MOLECULAR IMAGING PROBES BASED ON LOADED REACTIVE NANO-SCALE LATEX.”

### FIELD OF THE INVENTION

This invention relates generally to the cutaneous or transdermal administration into small animals or humans of compositions such as optical, single photon emission computed tomography (SPECT), multimodal, drug or biological cargo-laden nanoparticle(s).

### BACKGROUND OF THE INVENTION

Reference is made to the following commonly assigned, co-pending U.S. patent applications, the disclosures of which are incorporated by reference:

regular Ser. No. 11/221,530 filed on Sep. 9, 2005 by Vizard et al entitled “APPARATUS AND METHOD FOR MULTI-MODAL IMAGING”;

regular Ser. No. 11/400,935 filed on Apr. 10, 2006 by Harder et al. entitled “FUNCTIONALIZED POLY(ETHYLENE GLYCOL)”;

regular Ser. No. 11/732,424 filed on Apr. 3, 2007 by Leon et al. entitled “LOADED LATEX OPTICAL MOLECULAR IMAGING PROBES”;

regular Ser. No. 11/738,558 filed Apr. 23, 2007 by Zheng et al. entitled “IMAGING CONTRAST AGENTS USING NANOPARTICLES”;

regular Ser. No. 12/196,300 filed on Sep. 7, 2007 by Harder et al entitled “APPARATUS AND METHOD FOR MULTI-MODAL IMAGING USING NANOPARTICLE MULTI-MODAL IMAGING PROBES”;

regular Ser. No. 11/930,417 filed on Oct. 31, 2007 by Zheng et al. entitled “ACTIVATABLE IMAGING PROBE USING NANOPARTICLES”;

provisional Ser. No. 61/024,621 filed on Jan. 30, 2008 by Feke et al. entitled “APPARATUS AND METHOD FOR MULTIMODAL IMAGING”.

Electronic imaging systems are well known for enabling molecular imaging. An exemplary electronic imaging system 10 is shown in FIG. 1 and diagrammatically illustrated in FIG. 2. The illustrated system is the Image Station 4000MM Multimodal Imaging System available from the Carestream Health Inc. (refer to www.carestreamhealth.com). System 10 includes a light source 12, an optical compartment 14; an optional mirror 16 within compartment 14, a lens and camera system 18, and a communication and computer control system 20 which can include a display device, for example, a computer monitor. Camera and lens system 18 can include an emission filter wheel, not illustrated, for fluorescent imaging. Light source 12 can include an excitation filter selector, not illustrated, for fluorescent excitation or bright field color imaging. In operation, an image of an object is captured using lens and camera system 18 which converts the light image into an electronic image, which can be digitized. The digitized image can be displayed on the display device, stored in memory, transmitted to a remote location, processed to enhance the image, and/or used to print a permanent copy of the image. U.S. Pat. No. 7,031,084 of Vizard et al., the disclosure of which is incorporated herein by reference, gives an example of an electronic imaging system suitable for lens and camera system 18.

To increase the effectiveness of these electronic imaging systems, considerable effort has been focused upon developing nanoparticulate probes capable of delivering imaging agents directly to the cells of interest within a test animal, human or tissue sample. These nanoparticles are also capable of carrying biological, pharmaceutical or diagnostic agents into and within living organisms. These agents are typically comprised of drugs, therapeutics, diagnostics, biocompatibilization functionalities, contrast agents, and targeting moieties attached to or contained within a nanoparticulate carrier. Work in this field has the goals of affording imaging and therapeutic agents with such profound advantages as greater circulatory lifetimes, higher specificity, lower toxicity and greater therapeutic effectiveness. Work in the field of nanoparticulate assemblies has promised to significantly improve the treatment of cancers and other life threatening diseases and may revolutionize their clinical diagnosis and treatment.

Specific nanoparticles have been found to be nontoxic, and are capable of entry into small capillaries in the body, transport in the body to a disease site, crossing biological barriers (including but not limited to the blood-brain barrier and intestinal epithelium), absorption into cell endocytic vesicles, crossing cell membranes and transportation to the target site inside the cell. The particles in that size range are believed to be more efficiently transferred across the arterial wall compared to larger size microparticles, see Labhasetwar et al., Adv. Drug Del. Res. 24:63 (1997). Without wishing to be bound by any particular theory it is also believed that because of high surface to volume ratio, the small size is essential for successful targeting of such.

It would be desirable to produce multimodal biological targeting units or imaging probes comprising nanoparticles for use as carriers for bioconjugation and targeted delivery which are stable so that they can not only be injected in vivo, especially intravascularly, but be administered transdermally. Further, it would be desirable that the transdermally administered nanoparticles for use as carriers be stable under physiological conditions (pH 7.4 and 137 mM NaCl). Still further, it would be desirable that such transdermally administered particles avoid detection by the immune system.

In addition, for optical molecular imaging nanoparticles are needed that are less than 100 nm in size, resist protein adsorption, have convenient attachment moieties for the attachment of multimodal biological targeting units. These multimodal biological targeting units may contain emissive dyes that emit in the infrared (IR), near IR (NIR), are capable of being detected by and enhancing X-ray imaging, being detected by and enhancing magnetic resonance imaging (MRI) and being detected by and enhancing optical imaging.

Various nanoparticle probes presently are injected in vivo, especially intravascularly into both small animals for preclinical work and into humans for the diagnosis and treatment of such diseases as cancer, etc. It would be more desirable if these multimodal biological targeting units or imaging probes comprising nanoparticles could be administered cutaneously or more specifically delivered via a transdermal patch.

Currently many conventional pharmaceutical compositions are administered to humans by passive cutaneous routes, such as transdermal delivery from a patch applied to the skin. Examples of drugs that are routinely administered by this route are nitroglycerin, steroid hormones, and some analgesics (such as fentanyl). Transdermal administration avoids initial inactivation of drugs in the gastrointestinal tract, and provides continuous and accurately controlled dosages usually over a relatively short period of time (such as a day or week), without requiring active participation by the patient. Continuous sustained administration provides better bioavailability of the drug, without peaks and troughs.

U.S. Pat. No. 7,217,735 to Au et al discloses methods for enhancing delivery of therapeutic agents, such as macromolecules and drugs, into the interior of tissues, such as solid tissues or tumors by using an apoptosis inducing agent, such as paclitaxel, in doses which create channels within the tissues, and enhance the penetration of therapeutic agents to the interior of the tissue. Au, however does not teach using transdermal methods for introducing bio-laden nanoparticles designed for the purpose of optical molecular imaging of animals or humans.

U.S. Patent Application Publication 2007/0077286 by Ishihara et al. discloses an external preparation or injectable preparation that exerts the effect of enabling transdermal or transmucosal in vivo absorption of fat-soluble drugs and water-soluble drugs. Drug-containing nanoparticles (secondary nanoparticles) are provided by causing primary nanoparticles containing a fat-soluble drug or fat-solubilized water-soluble drug to act with a bivalent or trivalent metal salt. Ishihara does not teach using a transdermal method for introducing bio-laden nanoparticles designed for the purpose of optical molecular imaging of animals or humans.

U.S. Patent Application Publication 2006/0147509 by Kirkby et al. discloses compositions for transdermal delivery of at least one immunogen to an individual, via a patch applied the skin. An immunogen in the form of a Poslntro or an ISCOM may be delivered. Kirkby also teaches delivery of an immunogen with an occlusion vehicle in the form of a pressure sensitive adhesive and an immunogen delivery system comprising at least one saponin and at least one sterol. Kirkby does not teach using a transdermal method for introducing bio-laden nanoparticles designed for the purpose of optical molecular imaging of animals or humans.

None of the prior art teaches transdermal delivery of multimodal imaging nanoparticles or the transdermal delivery to a mouse or human for multimodal molecular imaging. Nor does the prior art disclose devices for adhering to the tail of a mouse for transdermal delivery, which avoids known vagaries of controlling injected amounts into the tail veins of test animals such as a mouse. It would be desirable to be able to accurately and quickly deliver an optical, SPECT, multimodal, drug or biological cargo-laden nanoparticle(s) cutaneously via a transdermal patch into small animals or humans.

### SUMMARY OF THE INVENTION

The device and method of the present invention will allow researchers in pharmaceutical, biotech companies, and academic setting to circumvent the invasive injection process of small animals. The invention will be particularly useful, when experiments or drug trials need tens or in some cases hundreds of small animals. Apart from the time saving process, the vital advantage is the uniformity in dose delivery when using the invention. The tail-vein injections are prone for lots of vagaries in the amounts injected.

The invention comprises both a method and a device for transdermal delivery to an animal or human of biological cargo-laden nanoparticles. The particles may include multimodal optical molecular imaging probes. The particles may be delivered by providing them in a form that can be absorbed through the skin and applying them to the skin of an animal or human. The application may be accomplished using biological cargo-laden nanoparticles in a device attachable to the skin. The device may be attached directly to the skin of a human by a patch containing a vasodilating agent or agents, a patch containing micro needles, or a patch containing multi-layer time release material. The device to be attached directly to the skin of an animal may be secured to the tail and contain a vasodilating agent or agents, or micro needles, or a multi-layer time release material. The biological cargo-laden nanoparticles may comprise drugs, vaccines, bio-pharmaceuticals, imaging contrast agents, multimodal imaging contrast agents, biomolecules, or anti-infectives. The device may include a first plurality of different types of biological cargo-laden nanoparticles located in a corresponding second plurality of separate time release layers.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 shows a perspective view of an exemplary electronic imaging system;

FIG. 2 shows a diagrammatic view of the electronic imaging system of FIG. 1;

FIG. 3A shows a diagrammatic side view of an imaging system suitable for use in accordance with the present invention;

FIG. 3B shows a diagrammatic front view of the imaging system of FIG. 3A;

FIG. 4 shows a perspective view of the imaging system of FIGS. 3A and 3B;

FIG. 5 is a diagrammatic view of a transdermal device according to the invention attached to the tail of a mouse;

FIG. 6 is an enlarged partial view of the transdermal device of FIG. 5, seen as attached in close proximity to the tail vein of the mouse;

FIG. 7 is a cross-sectional view of one embodiment of the transdermal device used in the present invention;

FIG. 8 is a schematic cross-section of the layers of the transdermal device taken across line 8-8 of FIG. 7;

FIG. 9 is a schematic cross-section illustrating one embodiment for protecting the contact surface of the transdermal device before use in accordance with the present invention;

FIG. 10 is a schematic illustrating a second embodiment for protecting the contact surface of the transdermal device before use in accordance with the present invention;

FIG. 11 is a schematic illustrating a first embodiment of a method for attaching the transdermal device to the tail of a mouse in accordance with the present invention;

FIG. 12 is a schematic illustrating a second embodiment of a method for attaching the transdermal device to the tail of a mouse in accordance with the present invention;

FIG. 13 is a schematic illustrating a third embodiment of a method for attaching the transdermal device to the tail of a mouse in accordance with the present invention;

FIG. 14 shows a diagrammatic partial view of the mouse in the sample chamber on the sample object stage of the imaging system of FIGS. 3A and 3B in accordance with the present invention;

FIG. 15 is a perspective and partial schematic view of a transdermal patch made in accordance with the present invention wherein the transdermal patch of FIG. 16 is placed on the surface of the skin;

FIG. 16 is a cross-sectional view of a portion of a transdermal patch;

FIG. 17 is a cross-sectional view of a portion of another embodiment of the transdermal device made in accordance with the present invention wherein the receiver comprises a multi-layer time-release material;

FIG. 18 is a cross-sectional view of a portion of yet another embodiment of the transdermal device made in accordance with the present invention that comprises a single layer time-release material, and

FIG. 19 is an electron micrograph close-up of microneedles, and

FIGS. 20A and B show the experimental results of noninvasive delivery of KODAK X-SIGHT nanospheres via a Nicoderm (trademark) patch.

### DETAILED DESCRIPTION OF THE INVENTION

The invention will be described in detail with particular reference to certain preferred embodiments thereof, but it will be understood that variations and modifications can be effected within the spirit and scope of the invention. Unless otherwise noted, technical terms are used according to conventional usage. Definitions of common terms in pharmacology may be found in Remington: The Science and Practice of Pharmacy, 19th Edition, published by Mack Publishing Company, 1995 (ISBN 0-912734-04-3). Transdermal delivery is discussed in particular at page 743 and pages 1577-1584. The singular forms “a,” “an,” and “the” refer to one or more than one, unless the context clearly dictates otherwise. The term “comprising” means “including.”

A “bioactive” material, composition, substance or agent is a composition which affects a biological function of a subject to which it is administered. An example of a bioactive material used to create a composition is a pharmaceutical substance, such as a drug, which is given to a subject to alter a physiological condition of the subject, such as a disease. Examples of bioactive materials that are capable of transdermal delivery include pharmaceutical compositions. As used herein, the terms “bioactive material” and/or “particles of a bioactive material” refer to any compound or composition of matter which, when administered to an organism (human or nonhuman animal) induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. More particularly, the term “bioactive material” includes compounds or compositions for use in all of the major therapeutic areas including, but not limited to, anti-infectives such as antibiotics and antiviral agents; analgesics and analgesic combinations; local and general anesthetics; anorexics; anti-arthritics; anti-asthmatic agents; anticonvulsants; antidepressants; antihistamines; anti-inflammatory agents; antinauseates; anti-migraine agents; antineoplastics; antipruritics; antipsychotics; antipyretics; antispasmodics; cardiovascular preparations (including calcium channel blockers, beta-blockers, beta-agonists and antiarrythmics); anti-hypertensives; diuretics; vasodilators; central nervous system stimulants; cough and cold preparations; decongestants; diagnostics; hormones; bone growth stimulants and bone resorption inhibitors; immunosuppressives; muscle relaxants; psycho stimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including double- and single-stranded molecules and supercoiled or condensed molecules, gene constructs, expression vectors, plasmids, antisense molecules and the like). Particles of a bioactive material, alone or in combination with other drugs or agents, are typically prepared as pharmaceutical compositions which can contain one or more added materials such as carriers, vehicles, and/or excipients.

“Carriers,” “vehicles” and “excipients” generally refer to substantially inert materials which are nontoxic and do not interact with other components of the composition in a deleterious manner. These materials can be used to increase the amount of solids in particulate pharmaceutical compositions. Examples of suitable carriers include silicone, gelatin, waxes, and like materials. Examples of normally employed “excipients,” include pharmaceutical grades of dextrose, sucrose, lactose, trehalose, mannitol, sorbitol, inositol, dextran, starch, cellulose, sodium or calcium phosphates, calcium sulfate, citric acid, tartaric acid, glycine, high molecular weight polyethylene glycols (PEG), erodible polymers (such as polylactic acid, polyglycolic acid, and copolymers thereof), and combinations thereof.

In addition, it may be desirable to include a charged lipid and/or detergent in the pharmaceutical compositions. Such materials can be used as stabilizers, anti-oxidants, or used to reduce the possibility of local irritation at the site of administration. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, e.g., TWEEN® surfactants (Atlas Chemical Industries, Wilmington, Del.), polyoxyethylene ethers, e.g., Brij, pharmaceutically acceptable fatty acid esters, e.g., lauryl sulfate and salts thereof (SDS), and like materials. Bioactive materials, compositions and agents also include other biomolecules, such as proteins and nucleic acids, or liposomes and other carrier vehicles that contains bioactive materials.

“Cutaneous” refers to the skin, and “cutaneous delivery” means application to the skin. This form of delivery can include either delivery to the surface of the skin to provide a local or topical effect, or transdermal delivery. The following terms are intended to be defined as indicated below. The term “transdermal” delivery refers to transdermal (or “percutaneous”), i.e., delivery by passage of a bioactive material through the skin. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987).

Researchers involved in the clinical testing of bioactive material compositions use tens to hundreds of small animals such as mice for these types experiments and most of these experiments involve some type of multimodal imaging of these animals. For multimodal imaging to be effective two elements are necessary. The first is a multimodal imaging system and the second is an imaging probe.

The type of imaging system described here is an example of a multimodal imaging system used by researchers to capture images using differing modes of imaging. This type of multimodal imaging system enables and simplifies multi-modal imaging allowing the relative movement of probes to be kinetically resolved over the time period that the animal is effectively immobilized (which can be tens of minutes). Alternatively, the same animal may be subject to repeated complete image analysis over a period of days/weeks required to assure completion of a pharmaceutical study, with the assurance that the precise anatomical frame of reference (particularly, the x-ray) may be readily reproduced upon repositioning the object animal.

Imaging modes supported by the multimodal imaging system include: x-ray imaging, bright-field imaging, dark-field imaging (including luminescence imaging, fluorescence imaging) and radioactive isotope imaging. Images acquired in these modes can be merged in various combinations for analysis. For example, an x-ray image of the object can be merged with a near IR fluorescence image of the object to provide a new image for analysis.

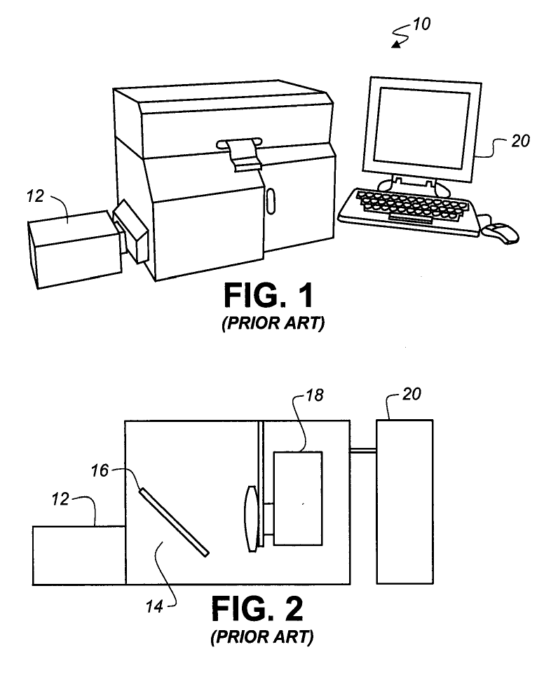

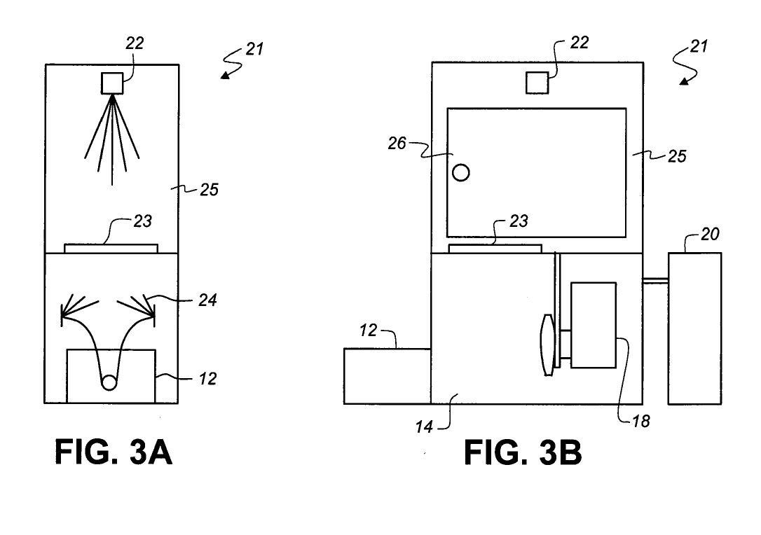

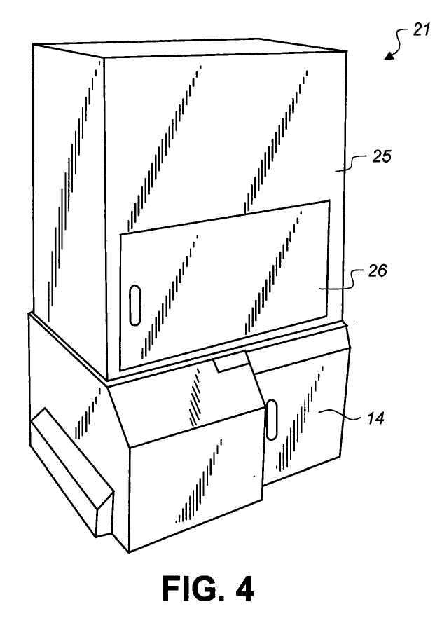

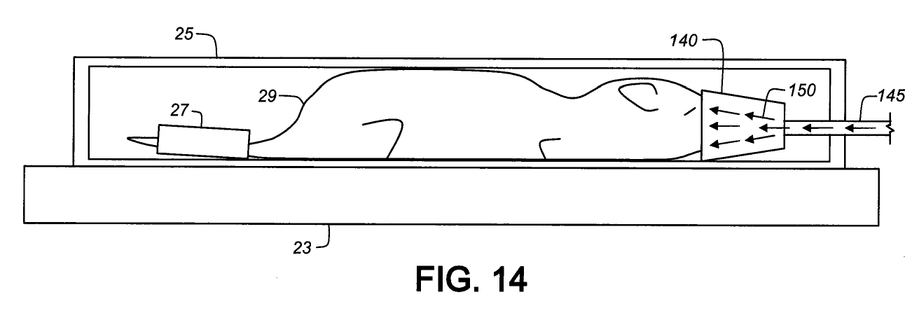

A multimodal imaging system suitable for use in accordance with the invention is illustrated in FIGS. 3A, 3B, and 4. System 21 includes the components illustrated in FIGS. 1 and 2. Also, as best shown in FIG. 3A, imaging system 21 includes an x-ray source 22 and a sample object stage 23. Imaging system 21 further comprises epi-illumination, for example, using fiber optics 24, which directs conditioned light of appropriate wavelength and divergence toward sample object stage 23 to provide bright-field or fluorescent imaging. Sample object stage 23 is disposed within a sample environment 25, which allows access to the object being imaged. Preferably, sample environment 25 is light-tight and fitted with light-locked gas ports for environmental control. Such environmental control might be desirable for controlled x-ray imaging or for support of particular specimens as shown in FIG. 14. Environmental control enables practical x-ray contrast below 8 Kev (air absorption) and aids in life support for biological specimens.

Imaging system 21 further includes an access means or member 26 to provide convenient, safe and light-tight access to sample environment 25. Access means are well known to those skilled in the art and can include a door, opening, labyrinth, and the like. Additionally, sample environment 25 is preferably adapted to provide atmospheric control for sample maintenance or soft x-ray transmission (e.g., temperature/humidity/alternative gases and the like). The inventions disclosed in previously mentioned U.S. patent application Ser. No. 12/196,300, Ser. No. 11/221,530 and provisional Ser. No. 61/024,621 are examples of electronic imaging systems capable of multimodal imaging and suitable for use in accordance with the present invention.

In order for multimodal imaging systems to be effective an imaging probe is needed. The “bioactive material” composition previously discussed may also include various agents that enhance or improve disease diagnosis. For example, an optical, SPECT, MRI, or multimodal imaging probe may be in the form of a biological cargo-laden nanoparticle(s).

To assemble the biological, pharmaceutical or diagnostic components to a described biological cargo-laden nanoparticle used as a carrier, the components can be associated with the nanoparticle carrier through a linkage. By “associated with”, it is meant that the component is carried by the nanoparticle. The component can be dissolved and incorporated in the nanoparticle non-covalently.

Generally, any manner of forming a linkage between a biological, pharmaceutical or diagnostic component of interest and a nanoparticle used as a carrier can be utilized. This can include covalent, ionic, or hydrogen bonding of the ligand to the exogenous molecule, either directly or indirectly via a linking group. The linkage is typically formed by covalent bonding of the biological, pharmaceutical or diagnostic component to the nanoparticle used as a carrier through the formation of amide, ester or imino bonds between acid, aldehyde, hydroxy, amino, halo-aromatic, or hydrozoa groups on the respective components of the complex. Art-recognized biologically labile covalent linkages such as imino bonds and so-called “active” esters having the linkage —COONR2, —O—O— or —COOC are preferred. The biological, pharmaceutical or diagnostic component of interest may be attached to the pre-formed nanoparticle or alternately the component of interest may be pre-attached to a polymerizeable unit and polymerized directly into the nanoparticle during the nanoparticle preparation. Hydrogen bonding, e.g., that occurring between complementary strands of nucleic acids, can also be used for linkage formation.

In the imaging probe as described in the previously mentioned U.S. patent application Ser. No. 11/401,343, the nanoparticles are in the form of a nanogel comprising a water-compatible, swollen, branched polymer network of repetitive, cross-linked, ethylenically unsaturated monomers of Formula I:

(X)m-(Y)n-(Z)o Formula I

wherein X is a water-soluble monomer containing ionic or hydrogen bonding moieties; Y is a water-soluble macromonomer containing repetitive hydrophilic units bound to a polymerizeable ethylenically unsaturated group; Z is a multifunctional cross-linking monomer; m ranges from 50-90 mol %; n ranges from 2-30 mol %; and o range from 1-15 mol %. The present invention also relates to a method for preparing a nanogel comprising preparing a header composition of a mixture of monomers X, Y, and Z, and a first portion of initiators in water, preparing a reactor composition of a second portion initiators, surfactant, and water sufficient to afford a composition of 1-10% w/w of monomers X, Y, and Z; bringing the reactor composition to the polymerization temperature; holding the reactor composition at the polymerization temperature for the duration of the reaction, and adding the header composition to the reactor composition over time to form a reaction mixture, wherein the nanogel comprises a water-compatible, swollen, branched polymer network of repetitive, cross-linked, ethylenically unsaturated monomers of Formula I:

(X)m-(Y)n-(Z)o Formula I

wherein m ranges from 50-90 mol %; n ranges from 2-30 mol %; and o range from 1-15 mol %. For the imaging probe to be multimodal the nanoparticle making up the probe must carry two or more imaging components for example a near IR dye for fluorescent imaging and gadolinium for x-ray imaging.

In the imaging probe as described in the previously mentioned U.S. patent application Ser. No. 11/732,424, a loaded latex particle may comprise a latex material made from a mixture represented by Formula II:

(X)m-(Y)n-(Z)o-(W)p, Formula II

wherein Y is at least one monomer with at least two ethylenically unsaturated chemical functionalities; Z is at least one polyethylene glycol macromonomer with an average molecular weight of between 300 and 10,000; W is an ethylenic monomer different from X, Y, or Z; and X is at least one water insoluble, alkoxethyl containing monomer; and m, n, o, and p are weight percent ranges of each component monomer, wherein m ranges between 40-90 percent by weight, n ranges between 1-10 percent by weight, o ranges between 20-60 percent by weight, and p is up to 10 percent by weight; and wherein said particle is loaded with a fluorescent dye.

In the imaging probe as described in the previously mentioned U.S. patent application Ser. No. 11/738,558, the nanoparticles are derived from self-assembly of amphiphilic block or graft copolymers to form crosslink particles with imaging dye immobilized in the particle, more specifically the imaging dye is immobilized via covalent chemical bond in the core of the nanoparticles and alkoxy silane cross-linking results in organic/inorganic hybrid materials.

It is well known that, in the presence of a solvent or solvent mixture that is selective for on block, amphiphilic block or graft copolymers have the ability to assemble into colloidal aggregates of various morphologies. In particular, significant interest has been focused on the formation of polymeric micelles and nanoparticles from amphiphilic block or graft copolymers in aqueous media. This organized association occurs as polymer chains reorganize to minimize interactions between the insoluble hydrophobic blocks and water. The resulting nanoparticles possess cores composed of hydrophobic block segments surrounded by outer shells of hydrophilic block segments. The core-shell structures of amphiphilic micellar assemblies have been utilized as novel carrier systems in the filed of drug delivery.

The amphiphilic copolymers that are useful in the present invention have a hydrophilic water soluble component and a hydrophobic component. Useful water soluble components include poly(alkylene oxide), poly(saccharides), dextrans, and poly(2-ethyloxazolines), preferably poly(ethylene oxide). Hydrophobic components useful in the present invention include but are not limited to styrenics, acrylamides, (meth)acrylates, lactones, lactic acid, and amino acids. Preferably, the hydrophobic components derived from styrenics and (meth)acrylates containing cross-linkable alkoxy silane groups. The imaging dyes contain functional groups that can react with the cross-linkable groups of the hydrophobic component and are immobilized in the core of the nanoparticles by covalent bonding. More specifically the imaging dyes contain alkoxy silane groups. Since the imaging dyes are immobilized in the nanoparticles, the quantum efficiency is enhanced. Suitable particles are described in the previously mentioned U.S. patent application Ser. No. 11/930,417.

In the imaging probe as described in the previously mentioned U.S. patent application Ser. No. 11/930,417, the nanoparticle may be in the form of an amine-modified silica nanoparticle, having a biocompatible polymer shell comprising amine functionalities. The core/shell particle has attached one or more fluorescent groups, polymer groups such as polyethylene glycol, targeting molecules, antibodies or peptides. Suitable particles are described in previously mentioned U.S. patent application Ser. No. 11/165,849. Especially preferred are silica nanoparticles having a near infrared fluorescent core and having attached to their surface, amine groups and/or polyethylene glycol. For example the biological cargo-laden nanoparticle(s) may be a nanoparticulate imaging probe comprising an oxide core, a biocompatible polymeric shell covalently attached to the oxide core, a dye that produces emissions in response to electromagnetic radiation, a quencher that quenches the emissions of the dye, and a cleavable peptide that covalently binds the probe to a component selected from the group consisting of the dye and the quencher, such that the component is liberated from the probe when the peptide is cleaved, wherein the probe has a size of less than 100 nm and the emission of the dye molecules is quenched when the component is bound to the probe and not quenched when the component is liberated from the probe.

In multimodal imaging probes the nanoparticle has one or more imaging components capable of being imaged by one or more imaging modes such as luminescence or fluorescent imaging component, X-ray and MRI.

The luminescence or fluorescent imaging component can be a near IR dye. Fluorophores include organic, inorganic or metallic materials that luminesce with including phosphorescence, fluorescence and chemo luminescence and bioluminescence. Examples of fluorophores include organic dyes such as those belonging to the class of naphthalocyanines, phthalocyanines, porphyrins, coumarins, oxanols, flouresceins, rhodamines, cyanines, dipyrromethanes, azadipyrromethanes, squaraines, phenoxazines; metals which include gold, cadmium selenides, cadmium telerides; and proteins such as green fluorescent protein and phycobiliprotein, and chemo luminescence by oxidation of luminal, substituted benzidines, substituted carbazoles, substituted naphthols, substituted benzthiazolines, and substituted acridans.

### MRI+Optical

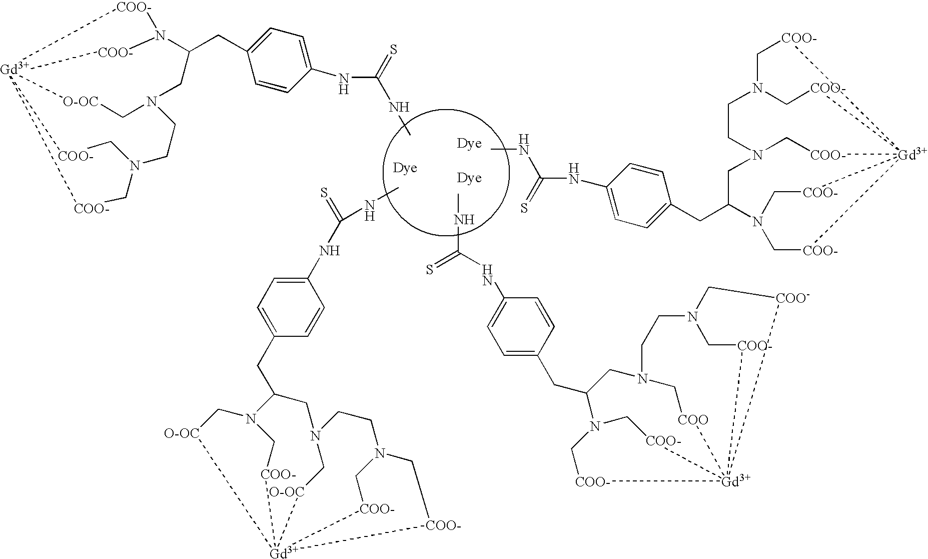

Where Dye is represented by the structure

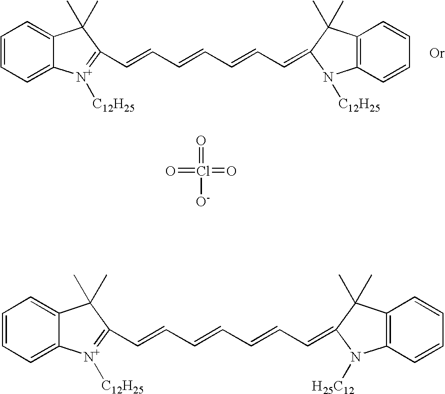

### MRI Contrast Agent

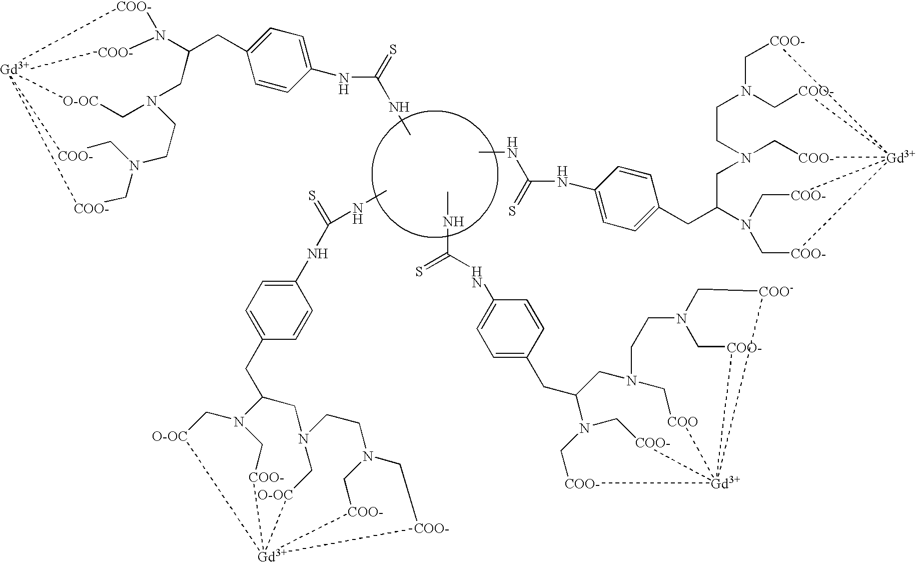

### Multimodal of Radioisotope and Dye

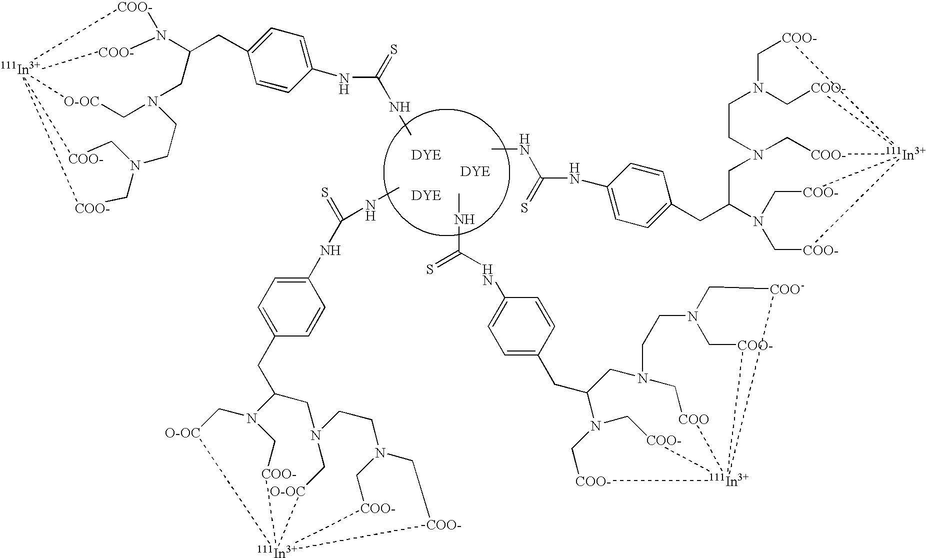

Where Dye is represented by the structure

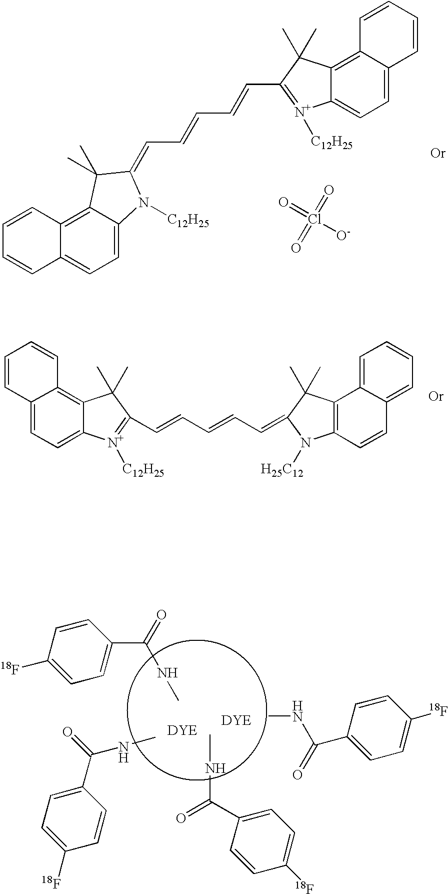

Where Dye is represented by the structure

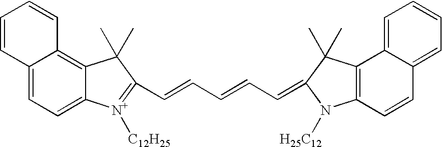

### Multimodal for X-Ray and Optical

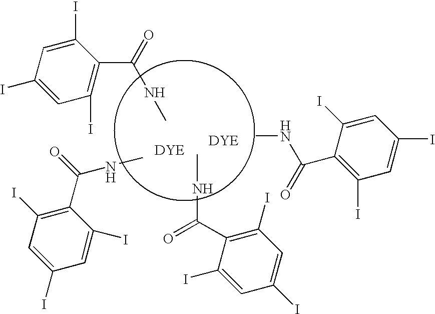

Where Dye is represented by the structure:

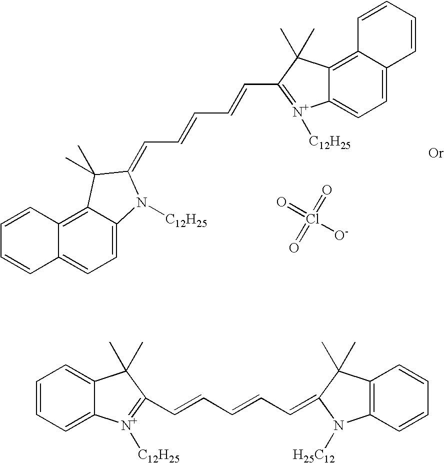

### X-Ray Contrast Agent

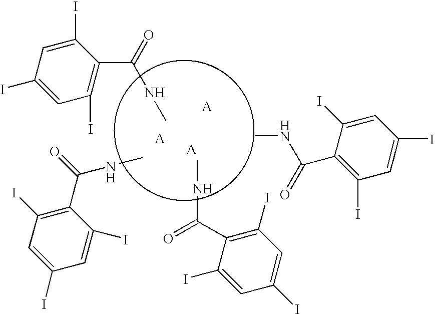

### Where A=

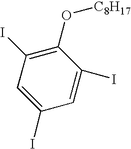

In the imaging probe as described in previously mentioned U.S. patent application Ser. No. 12/221,839 filed Aug. 7, 2008, a biological cargo-laden nanoparticle(s) may be a loaded reactive latex particle comprising a cross-linked polymer presented in Formula 1, wherein said cross-linked polymer comprises at least 45% water insoluble monomer and 1˜30 wt % monomer with reactive halo-aromatic conjugating group, and is loaded with molecular imaging agents of Formula III,

(X)m-(Y)n-(V)q-(T)o-(W)p Formula III

where m may range from 40-80 wt %, n may range from 1-10 wt %, q may range from 1-30 wt %, o may range from 10-60 wt %, and p is up to 10 wt %, where X is a water-insoluble, alkoxyethyl-containing monomer presented in Formula IV, where R1 is methyl or hydrogen, and R2 is an alkyl or aryl group containing up to 10 carbons,

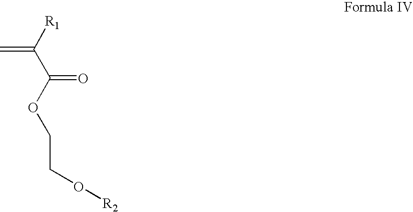

where Y is at least one monomer containing two ethylenically unsaturated chemical functionalities; W is an ethylenic monomer different from X, Y, V, or T; “V” is apolyethyleneglycol-methacrylate derivative (shown in Formula V), wherein n is greater than 1 and less than 130, preferably from 5 to 110 and CG is selected from 4-halo-3-nitrobenzoate, 2-halo-3-nitrobenzoate, 2-halo-4-nitrobenzoate, 4-halo-2-nitrobenzoate, 2-halo-5-nitrobenzoate, 3-halo-2-nitrobenzoate, 2-halonicotinate, 4-halonicotinate, 6-halonicotinate 2-haloisonicotinate, and 3-haloisonicotinate, where halo is selected from fluoro, chloro, bromo, and iodo;

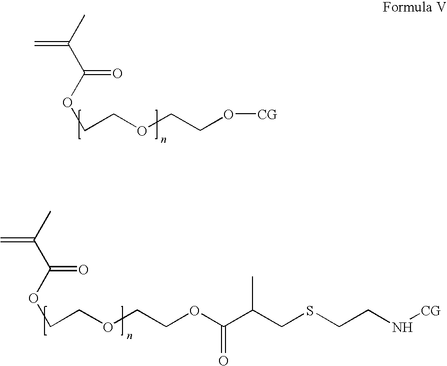

Formula V Chemical Structure of Monomer V
   where T is a polyethyleneglycolacrylate containing macromonomer presented in Formula VI in which

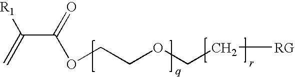

Formula VI Chemical Structure of Monomer T,

where R1 is hydrogen or methyl, q is 5-220, r is 1-10, and RG is a hydrogen or functional group.

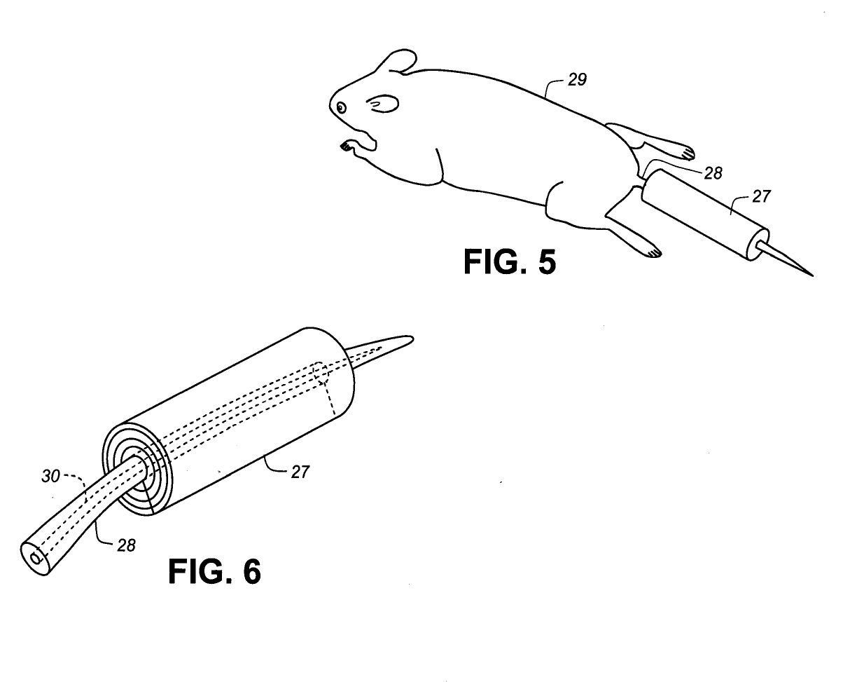

At present the primary method for administering these biological cargo-laden nanoparticle(s) is via tail-vein injections. This method of administration is both time consuming and subject to problems such as the control of the amount of bioactive material delivered. Accordingly, the present invention is directed at both a device and method for delivery of bioactive materials (biological cargo-laden nanoparticle(s)) in a controlled active, passive or timed manner. Now referring to FIG. 5, a transdermal device 27 is shown attached to the tail 28 of a mouse 29. In the preferred embodiment shown in the enlarged view of FIG. 6, transdermal device 27 is secured to tail 28 thereby placing transdermal device 27 in close proximity to the mouse's tail vein 30.

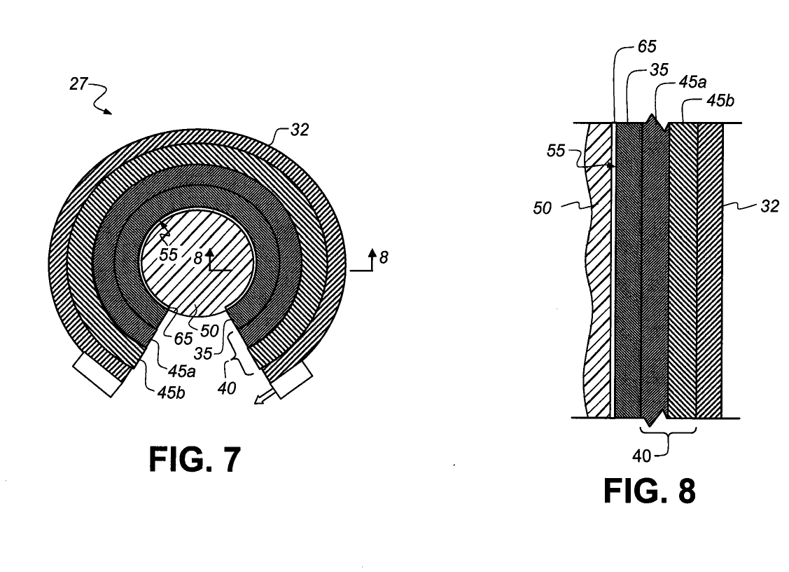

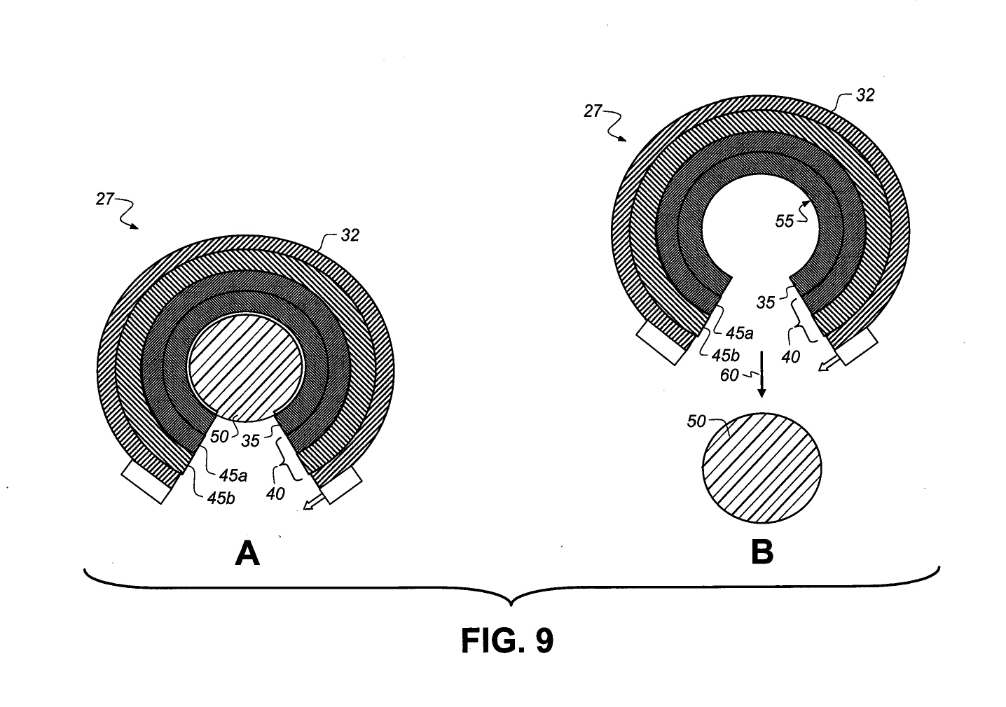

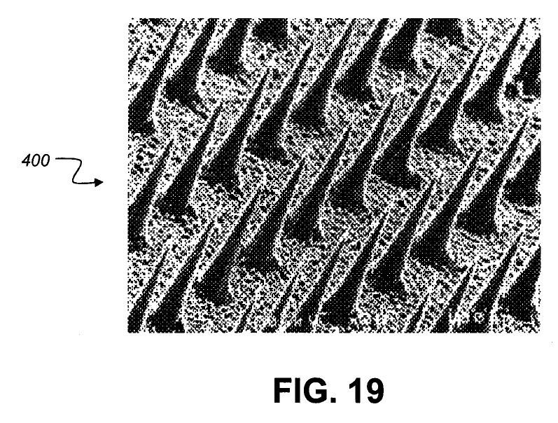

Referring now to the cross-sectional view illustrated in FIGS. 7 and 8, transdermal device 27 comprises multiple layers; a bite proof protective cover 32, an inner layer 35 which may contain an adhesive, an absorbent section 40 comprising one or more absorbent layers, for example 45a and 45b, which contain the bioactive material such as the biological cargo-laden nanoparticle(s) mixed with a vasodilating agent or agents selected from a list of: Nicotinic acid, nitotinate esters, papaverine, glyceryl trinitrate, lidocane, linsdomine, nicardipine, capronium chloride, acetylcholine 42, Nicotine and its analogs, and a core 50. Bite proof protective cover 32 may be made of materials such as silicone, polyethylene, polyvinylchloride, ABS, PVC, polycarbonate, HDPE (high density polyethylene), Kraton® (Kraton Polymers U.S. LLC, Houston, Tex.), PeBax® (Arkema, Inc., Philadelphia, Pa.) Plexiglass® (Arkema, Inc., Philadelphia, Pa.), polyacetalsDelrin® (E. I. du Pont de Nemours and Company, Wilmington, Del.) metal or polyurethane. Core 50 is located within layer 35 and may be made of material such as silicone, polyethylene, polyvinylchloride, ABS, PVC, polycarbonate, HDPE, Kraton®, PeBax® Plexiglass®, Delrin®, or polyurethane. Core 50 acts to preserve the integrity of a contact surface 55 of inner layer 35. Just prior to use, core 50 is removed as indicated by arrow 60 in FIG. 9, thus exposing contact surface 55. Contact surface 55 may comprise an array of microneedles as shown and later described with regard to FIG. 19.

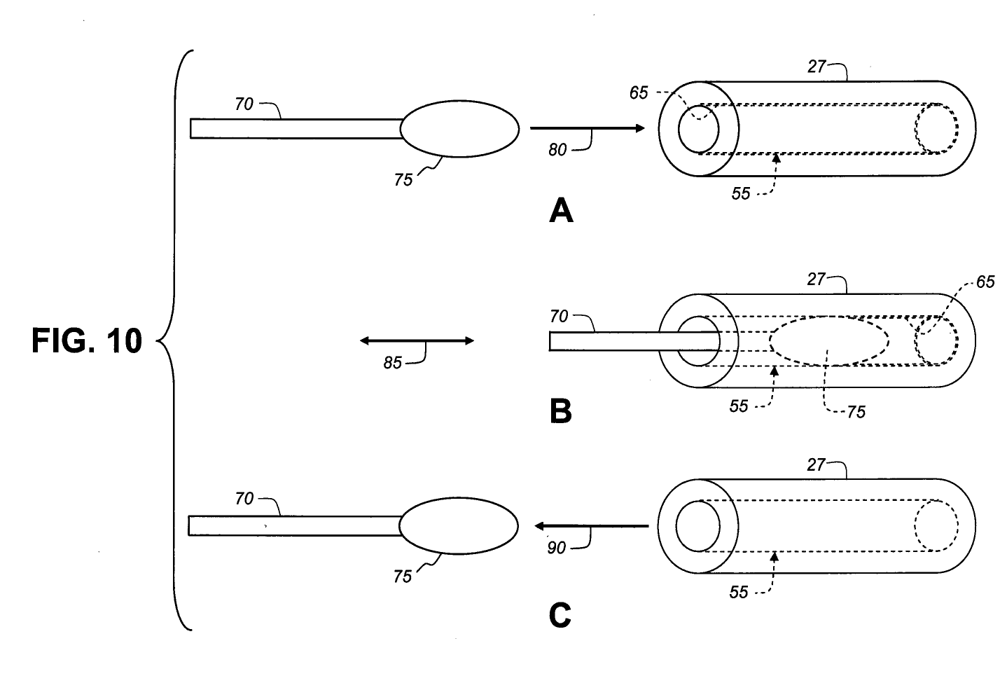

In lieu of, or in addition to, core 50 an inner protective layer 65, also shown in FIGS. 7 and 8, may be used to protect contact surface 55. When inner protective layer 65, which may be a gelatin, is present as illustrated in FIG. 10, it is removed by a swab 70 using the following steps: in step “A”, swab 70 whose tip 75 contains a liquid such as distilled water or a saline solution is first removed from its container (not shown) and inserted into transdermal device 27 as indicated by arrow 80. In step “B” swab tip 75 is then moved back and forth as indicated by arrow 85 removing inner protective layer 65, thus exposing contact surface 55. In step “C” swab tip 75 is removed from transdermal device 27 as indicated by arrow 90. Transdermal device 27 in now ready for insertion onto mouse's tail 28.

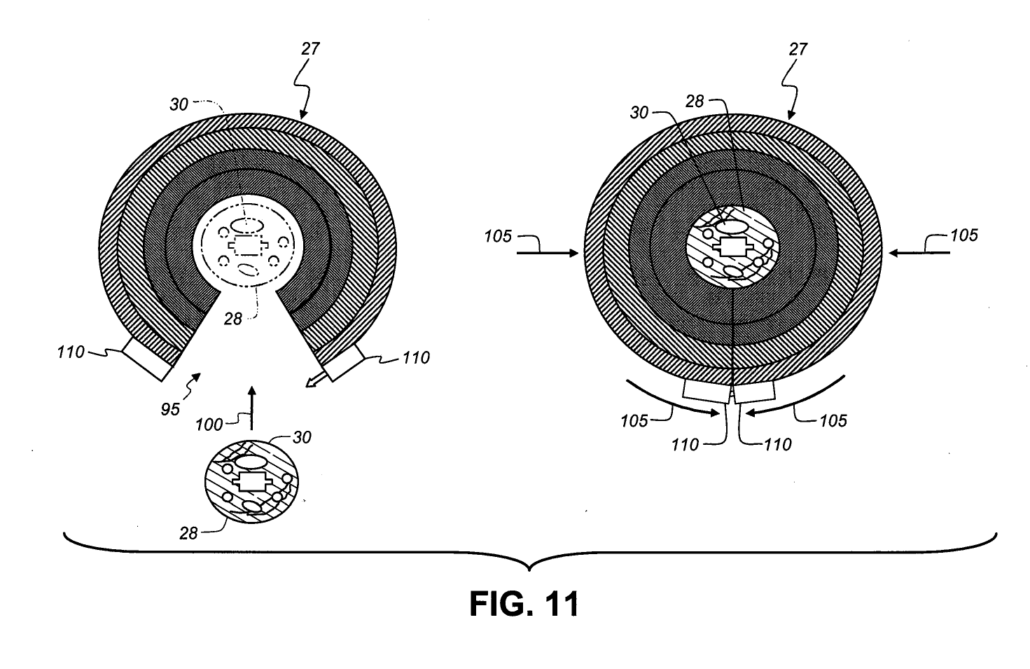

A first embodiment of the method for attaching transdermal device 27 is shown in FIG. 11. Transdermal device 27 is positioned onto mouse 29 by sliding tail 28 into transdermal device 27 through a slot 95 as indicated by arrow 100. Transdermal device 27 is then clamped onto mouse's tail 28 by pressing device 27 closed as indicated by arrows 105 until the two halves of a latch 110 snap together insuring the absorbent section 40 containing the biological cargo-laden nanoparticle(s) mixed with a vasodilating agent or agents 42 comes into intimate contact with tail 28.

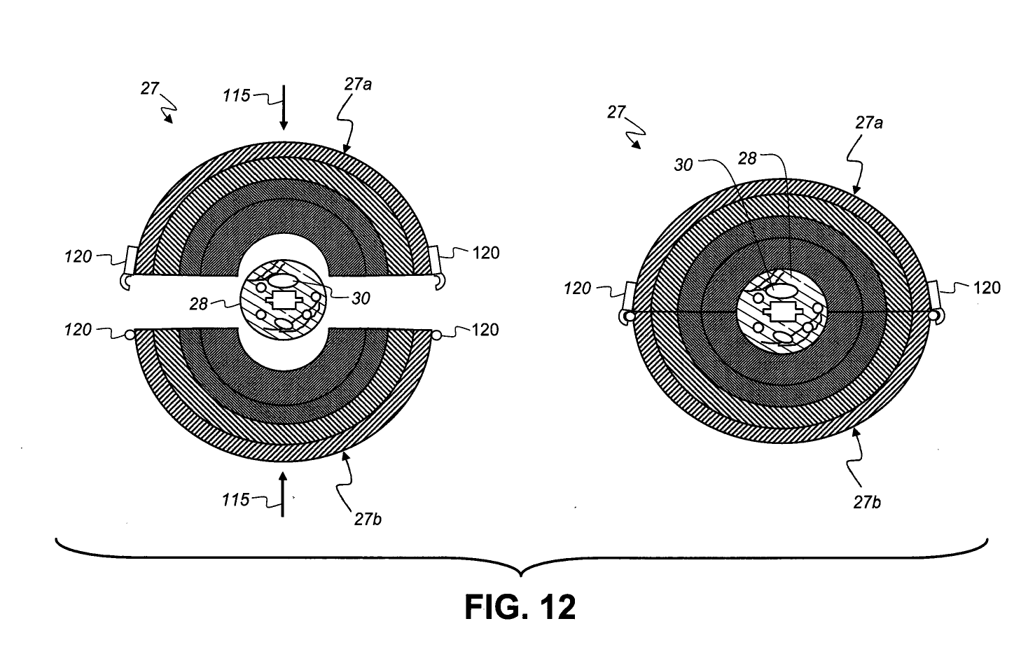

A second embodiment of the method for attaching transdermal device 27 is shown in FIG. 12. Transdermal device 27 is formed in two halves 27a, 27b that are positioned onto mouse 29 by placing tail 28 between the halves and snapping the two together as indicated by arrows 115. Transdermal device 27 is then clamped onto tail 28 by pressing halves 27a, 27b closed as indicated by arrows 115 until two latches 120 snap together insuring absorbent section 40 containing the biological cargo-laden nanoparticle(s) mixed with a vasodilating agent or agents 42 comes into intimate contact with tail 28.

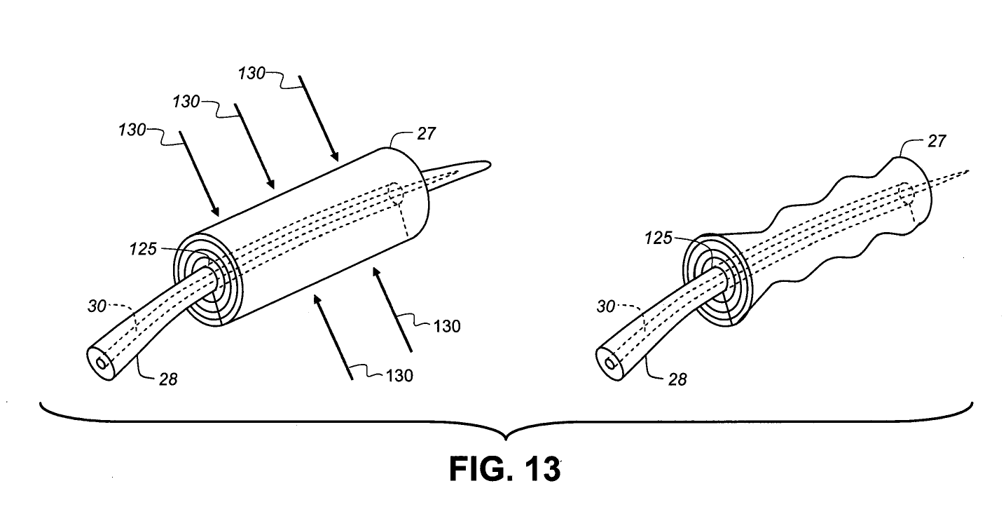

A third embodiment of the method for attaching transdermal device 27 is shown in FIG. 13. Transdermal device 27 is made with an outer protective cover 32 formed from a malleable fluoroplastic material such as polytetrafluoroethylene (PTFE, commonly called TFE), fluorinated ethylene propylene (FEP), perfluoroalkoxy (PFA), polychlorotrifluoroethylene (CTFE), poly (ethylene-chlorotrifluoroethylene (ECTFE) copolymer, ethylene tetrafluoroethylene (ETFE), polyvinylidene fluoride (PVDF), or polyvinylfluoride (PVF). Device 27 is positioned onto mouse 29 by sliding tail 28 into a central bore 125 in device 27 and gently squeezing transdermal device 27 to mold the device securely around tail 28 as indicated by the arrows 130, thus insuring absorbent section 40 containing the biological cargo-laden nanoparticle(s) mixed with a vasodilating agent or agents 42 comes into intimate contact with tail 28.

FIG. 14 shows a diagrammatic partial view of a sample chamber 25 and sample object stage 23 of imaging system 21 of FIGS. 3A and 3B. Once placed in chamber 25, mouse 29 is administered anesthesia through a respiratory device such as a nose cone or mask 140 connected to an outside source via a tube 145 which enters the chamber 25 via the light-locked gas ports. The anesthesia represented by the arrows 150 sedates the mouse through out the procedure.

In addition to using mice as test subjects, researchers also use larger animals such as rabbits, pigs, goats etc. in their experiments. When larger animals are used, the bioactive materials typically have been administered intravascularly by injection. Again it would be very advantageous to allow researchers in pharmaceutical, biotech companies, and academic setting to circumvent the invasive injection process with the use of transdermal delivery of these bioactive materials. The same is of course true of administering these bioactive materials to humans.

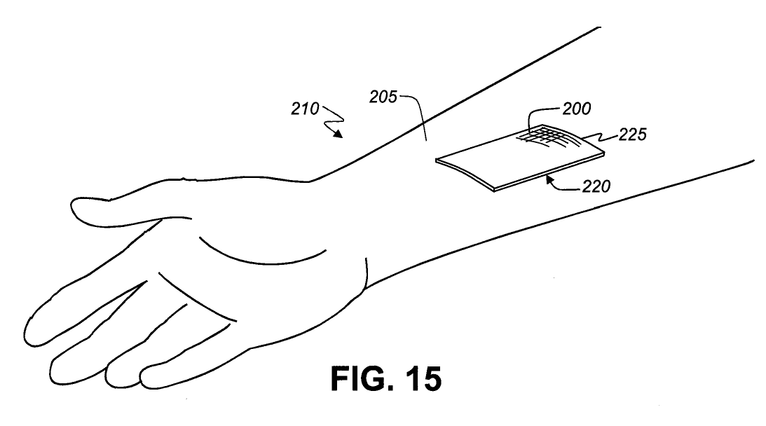

In using a transdermal device to administer the bioactive material, for example the imaging probe in the form of the biological cargo-laden nanoparticle as previously described, to a rabbit, the transdermal device maybe in the form of a patch applied directly to the skin surface. When applying the patch to the animal, the fur or hair is usually removed for example by shaving. In the example illustrated in FIG. 15, a transdermal patch 200 containing the biological cargo-laden nanoparticle(s) mixed with a vasodilating agent or agents, as previously described, is applied directly to the skin surface 205 of a human's arm 210. The patches with deliverable cargo can be administered to an animal or humans, in the close vicinity of a “target for action” such as a visible tumor, surface tumors, wounds, pathological organs, or other previously diagnosed tissue. This would facilitate the enrichment of the cargo at the “target site” with (i) minimal loss, (ii) minimal degradation, (iii) no unwanted physiological insults, (iv) minimal modifications, and so on.

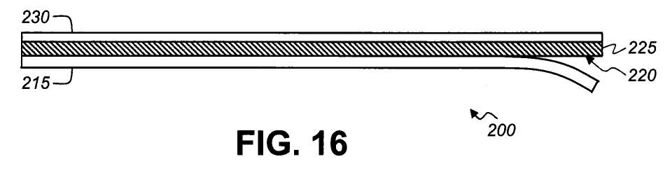

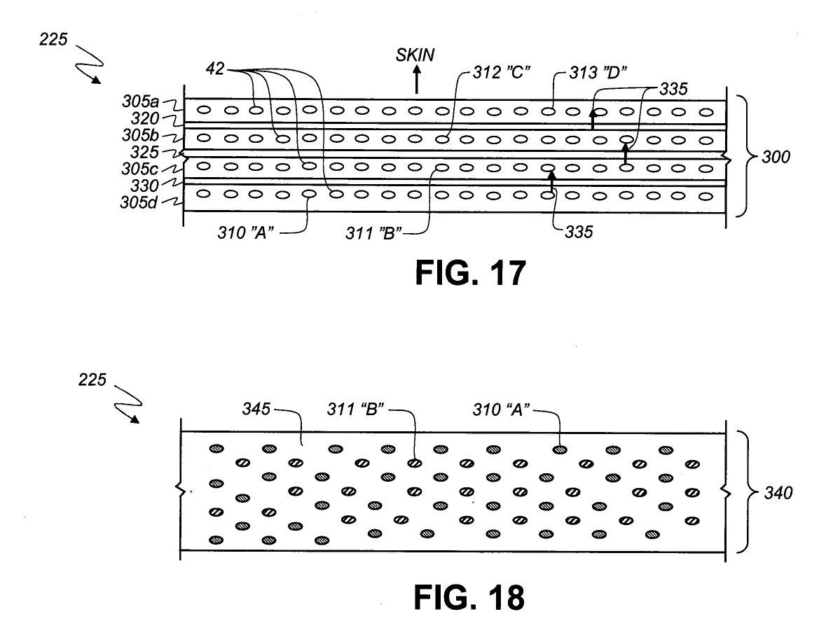

Transdermal patch 200 as illustrated in FIG. 16 comprises multiple layers, including a protective layer 215, such as a layer of a liquid impermeable thin polyester removable to expose an under surface 220 (that will contact skin 205 and may have adhesive strips not shown) of an absorbent layer or receiver layer 225. A fabric or other absorbent material may form receiver layer 225 which contains the bioactive materials such as the biological cargo-laden nanoparticle(s) mixed with a vasodilating agent 42 (to be described in FIGS. 17 and 18). Layer 225 may be adhered to skin surface 205 via under surface 220 as shown in FIG. 15. Finally, patch 200 may include an upper protective layer 230, also made of a liquid impermeable thin polyester. In one particular embodiment, the bioactive material 42 such the optical, SPECT, multimodal, drug or biological cargo-laden nanoparticle(s) may be mixed with vasodilators selected from a list of: Nicotinic acid, nitotinate esters, papaverine, glyceryl trinitrate, lidocane, linsdomine, nicardipine, capronium chloride, and acetylcholine. The mixture is contained in absorbent layer 225 of patch 200, and when applied to skin 205 allows skin 205 to gradually absorb the bioactive material from patch 200 in an accurate and uniform manner.

Any of the many types of transdermal patches may be used, or modified for use with the delivery system. For example the Testoderm® transdermal system (Alza Pharmaceuticals) uses a flexible backing of transparent polyester, and a testosterone containing film of ethylene-vinyl acetate copolymer membrane that contacts the skin surface and controls the rate of release of active agent from the system. The surface of the drug containing film is partially covered by thin adhesive stripes of polyisobutylene and colloidal silicon dioxide, to retain the drug film in prolonged contact with the skin.

FIG. 17 illustrates an embodiment of the absorbent layer 225, where absorbent layer 225 is a multi-layer time-release material 300. In the example illustrated in FIG. 17 there are four separate time release layers 305a, 305b, 305c, and 305d. To achieve time release, transdermal device 27 or patch 200 delivers the bioactive material such as biological cargo-laden nanoparticle(s) 42 as previously described, due to the location of each individual biological cargo-laden nanoparticle 310, 311, 312, and 313 in its appropriate, separate time release layer 315a, 315b, 315c, and 315d. Multi-layer time-release material 300 comprises several individual layers. The diffusivity of the drugs into and through each of the layers can be controlled by the type of material in each layer. Where one material such as cross-linked gelatin is used for all the time release layers 305a, b, c, and d, semipermeable layers 320, 325 and 330 may be placed between each of the time release layers to control the diffusion rate of the time release biological cargo-laden nanoparticles 310 “A”, 311 “B”, 312 “C” and 313 “D” through the layers 315a, b, c, and d respectively. Semipermeable layer 320 is permeable to biological cargo-laden nanoparticles 311 “B”, 312 “C” and 313 “D” but not permeable to biological cargo-laden nanoparticle 310 “A”. Likewise semipermeable layer 325 is permeable to biological cargo-laden nanoparticles 312 “C” and 313 “D” but not permeable to biological cargo-laden nanoparticle 311 “B”; and semipermeable layer 330 is permeable to biological cargo-laden nanoparticle 313 “D” but not permeable to biological cargo-laden nanoparticle 312 “C”. Using the semipermeable layers, the timed diffusion of the time release biological cargo-laden nanoparticles 310 “A”, 311 “B”, 312 “C” and 313 “D” to the skin can be controlled as indicated by the arrows 335.

For an example, multi-layer time-release material 300 may comprise time release layers dextran-bisacrylamide hydrogel (305a), dextran-methacrylate hydrogel (305b), carboxylmethyl dextran hydrogel (305c), and divinyl benzene-methacrylic acid hydrogel (305d), respectively, with thickness from 10 μm to 200 μm for each layer. The nanoparticles in each layer may be KODAK X-sight nanospheres 761 (nanoparticle 313 “D” in layer 305a), X-sight nanospheres 691 (nanoparticle 312 “C” in layer 305b), loaded reactive nanoscale latex particle (nanoparticle 311 “B” in layer 305c) and cross-linked organic-inorganic hybrid nanoparticle (nanoparticle 310 “A” in layer 305d).

FIG. 18 illustrates another embodiment of absorbent layer 225, where a transdermal delivery system comprises a single layer time-release material 340. Absorbent layer 225 includes a single layer of adhesive 345, such as DURO-TAK® (National Adhesives, Bridgewater, N.J.), serving also as a carrier for the bioactive material such as the biological cargo-laden nanoparticles 42 or 310 “A” and 311 “B”. In applications in which the biological cargo-laden nanoparticle(s) 42 is to be distributed to the host in a time release fashion, the adhesive-time release carrier material 340 controls, or assists in the control of the migration of the biological cargo-laden nanoparticle(s) 42 through the single layer into the host. The time release carrier materials 340 may be polymer-based hydrogels, including dextran hydrogel, dextran-methacrylate hydrogel, carboxyl methyl dextran hydrogel, dextran-polylactide hydrogel, poly(vinyl alcohol) hydrogel, heparin-poly(ethylene glycol)-poly(vinyl alcohol) hydrogel, poly(acrylic acid) hydrogel, divinylbenzene-methacrylic acid copolymer hydrogel, polyacrylamide hydrogel, acrylamide-bisacrylamide copolymer hydrogel, silicone hydrogel. In another embodiment both biological cargo-laden nanoparticle 310 “A” and biological cargo-laden nanoparticle 311 “B” could also be put in one layer, or biological cargo-laden nanoparticle 310 “A” could be put in layer two (not shown) and mitigate its delivery by controlling its diffusion through layer one. Any one of these methods could be used to control the diffusion of the bioactive particle or drug to achieve the appropriate time release. One layer may choose from dextran or its modified hydrogels, such as dextran hydrogel, dextran-methacrylate hydrogel, carboxylmethyl dextran hydrogel, dextran-polylactide hydrogel. The second layer may be cross-linked acrylic acid polymer hydrogels or vinyl alcohol containing hydrogels, including poly(acrylic acid) hydrogel, divinylbenzene-methacrylic acid copolymer hydrogel, poly(vinyl alcohol) hydrogel, heparin-poly(ethylene glycol)-poly(vinyl alcohol) hydrogel.

In yet another embodiment the surface of the transdermal patch or device that comes in direct contact with the skin of the large animal, human or the tail of the mouse may be comprised of an array of microneedles 400a shown in the electron micrograph of FIG. 19. As described in a paper published in the Nov. 17, 2003 online issue of the journal Proceedings of the National Academy of Sciences, microneedles have been developed for transdermal drug delivery providing controlled delivery across the skin. These needles increase skin permeability to macromolecules and nanoparticles up to 50 nm in radius. The microneedles penetrate the outer layer of skin known as the stratum corneum, carrying the bioactive materials such as optical, SPECT, multimodal, drug or biological cargo-laden nanoparticle(s) into deeper areas of the skin where they diffuse and are absorbed by capillaries which carry them into the bloodstream significantly increasing absorption of the bioactive materials through the skin. For example a microneedle carrier may consist of solid or hollow silicon microneedle arrays 10 millimeters square containing 400 needles ranging in size from one to 1,000 microns. by intra-peritoneal injection, ingestion or gavage These microneedle arrays may be also fabricated from metal and polymer materials that have sufficient strength to reliably penetrate the skin without breakage.

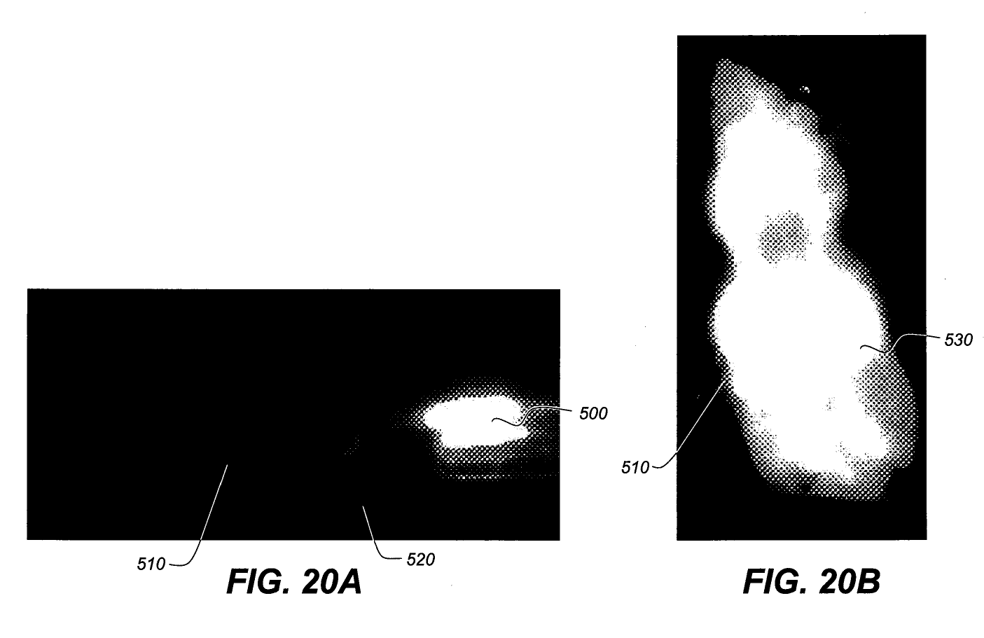

FIGS. 20A and B show the experimental results of noninvasive delivery of KODAK X-SIGHT 761 nanospheres via a Nicoderm (trademark) patch through a subject mouse's tail. In an experiment KODAK X-SIGHT 761 nanospheres were applied to the interior adhesive contact surface of a Nicoderm® patch 500. The patch was then applied to the tail of the subject mouse 510 and a near infrared fluorescent image was taken of the mouse 510 at time O-minutes using the imaging system 21 described in FIGS. 3A, 3B and 4. The near infrared fluorescent image 520 of the KODAK X-SIGHT nanospheres taken at time O-minutes can be seen in FIG. 20A. At a later time, time 3-hours using the imaging system 21, a second near infrared fluorescent image 530 was taken of the mouse 510. The resulting near infrared fluorescent image 530 is shown in FIG. 20B. In the near infrared fluorescent image 530 both the liver and kidneys of the mouse 510 show robust X-Sight 761 nanosphere signals. The experiment clearly demonstrates that the imaging agent X-Sight 761 nanospheres has successfully been transdermally delivered to the subject mouse 510 via the mouse's tail.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

### PARTS LIST

10 multimodal imaging system
12 light source
14 optical compartment
16 mirror
18 lens/camera system
20 control system
21 imaging system
22 x-ray source
23 sample object stage
24 fiber optics
25 sample environment
26 access means/member
27 transdermal device
27a, b one half of a transdermal device
28 tail
29 mouse 30 tail vein
32 protective cover
35 inner layer
40 absorbent section
42 bioactive material
45a, b absorbent layer
50 core
55 contact surface
60 arrow
65 inner protective layer
70 swab
75 swab tip
80 arrow
85 arrow
90 arrow
95 slot
100 arrow
105 arrow
110 latch
115 arrow
120 latch
125 central bore
130 arrow
140 respiratory device, nose cone or mask
145 tube
150 arrows
200 transdermal patch
205 skin
210 arm
215 removable protective layer
220 under surface
225 absorbent layer or receiver
230 upper protective layer
300 time release material
305a, b, c, d time release layers
310 bioactive particle
311 bioactive particle
312 bioactive particle
313 bioactive particle
320 semipermeable layer
325 semipermeable layer
330 semipermeable layer
335 arrow
340 single layer time-release material
345 single layer of adhesive
400 microneedle array
500 patch
510 subject
520 near infrared fluorescent image
530 near infrared fluorescent image

What is claimed is:

1. A method for transdermal delivery of biological cargo-laden nanoparticles, said particles including multimodal optical molecular imaging probes, to an animal or human, comprising steps of:
providing the biological cargo-laden nanoparticles in a form that can be absorbed through the skin; and
delivering said biological cargo-laden nanoparticles to said skin of an animal or human.

2. The method according to claim 1 wherein said delivering step is accomplished using biological cargo-laden nanoparticles in a device attachable to said skin.

3. The method according to claim 2 wherein said device is attached directly to said skin of a human by one of the following:
a patch containing a vasodilating agent or agents,
a patch containing micro needles, or
a patch containing multi-layer time release material.

4. The method according to claim 2, wherein said device is attached to said skin of an animal by one of the following:
a device secured to the tail containing a vasodilating agent or agents,
a device secured to the tail containing micro needles, or
a device secured to the tail containing multi-layer time release material.

5. The method according to claim 1, wherein said biological cargo-laden nanoparticles comprise any one of the following:
drugs,
vaccines,
biopharmaceuticals,
imaging contrast agents,
multimodal imaging contrast agents,
biomolecules, or
anti-infectives.

6. The method according to claim 1, further comprising steps of:
providing a support member adapted to receive said animal or human in an immobilized state,
delivering transdermally an imaging agent in the form of said biological cargo-laden nanoparticles to said animal or human, and
imaging said immobilized animal or human in a multimodal imaging system.

7. The method according to claim 6 wherein said imaging comprises use of any one of the following imaging modalities:
X-ray, or
near infrared fluorescent.

8. The method according to claim 1 wherein said biological cargo-laden nanoparticles include a loaded nanogel comprising a water-compatible, swollen, branched polymer network of repetitive, cross-linked, ethylenically unsaturated monomers represented by the formula:
(X)m-(Y)n-(Z)o
wherein X is a water-soluble monomer containing ionic or hydrogen bonding moieties; Y is a water-soluble macromonomer containing repetitive hydrophilic units bound to a polymerizeable ethylenically unsaturated group; Z is a multifunctional cross-linking monomer; m ranges from 50-90 mol %; n ranges from 2-30 mol %; and o range from 1-15 mol %.

9. The method according to claim 1 wherein said nanoparticles include a loaded latex particle comprising a latex material made from a mixture represented by formula:
(X)m-(Y)n-(Z)o-(W)p,
wherein Y is at least one monomer with at least two ethylenically unsaturated chemical functionalities; Z is at least one polyethylene glycol macromonomer with an average molecular weight of between 300 and 10,000; W is an ethylenic monomer different from X, Y, or Z; and X is at least one water insoluble, alkoxethyl containing monomer; and m, n, o, and p are weight percent ranges of each component monomer, wherein m ranges between 40-90 percent by weight, n ranges between 1-10 percent by weight, o ranges between 20-60 percent by weight, and p is up to 10 percent by weight; and wherein said particle is loaded with a fluorescent dye.

10. The method according to claim 1 wherein said biological cargo-laden nanoparticles include a loaded reactive latex particle comprising a cross-linked polymer represented by the following Formula 1, wherein said cross-linked polymer comprises at least 45% water insoluble monomer and 1˜30 wt % monomer with reactive halo-aromatic conjugating group, and is loaded with molecular imaging agents,
(X)m-(Y)n-(V)q-(T)o-(W)p Formula 1
where m may range from 40-80 wt %, n may range from 1-10 wt %, q may range from 1-30 wt %, o may range from 10-60 wt %, and p is up to 10 wt %.
where X is a water-insoluble, alkoxyethyl-containing monomer represented by the following Formula 2, where R1 is methyl or hydrogen, and R2 is an alkyl or aryl group containing up to 10 carbons, 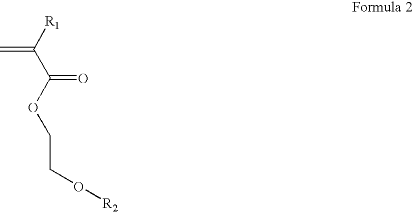
where Y is at least one monomer containing two ethylenically unsaturated chemical functionalities; W is an ethylenic monomer different from X, Y, V, or T; “V” is apolyethyleneglycol-methacrylate derivative represented by the following Formula 3, wherein n is greater than 1 and less than 130, preferably from 5 to 110 and CG is selected from 4-halo-3-nitrobenzoate, 2-halo-3-nitrobenzoate, 2-halo-4-nitrobenzoate, 4-halo-2-nitrobenzoate, 2-halo-5-nitrobenzoate, 3-halo-2-nitrobenzoate, 2-halonicotinate, 4-halonicotinate, 6-halonicotinate 2-haloisonicotinate, and 3-haloisonicotinate, where halo is selected from fluoro, chloro, bromo, and iodo; 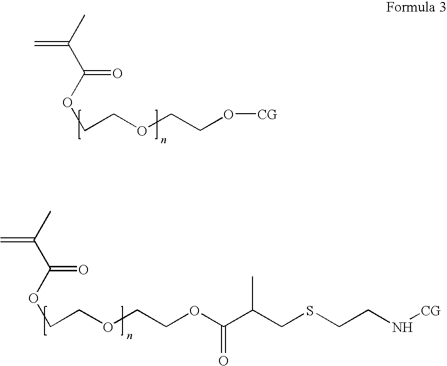
Formula 3 Chemical Structure of Monomer V,
where T is a polyethyleneglycolacrylate containing macromonomer represented by the following Formula 4 in which 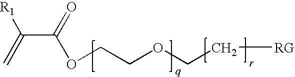
Formula 4 Tunable Structure,
where R1 is hydrogen or methyl, q is 5-220, r is 1-10, and RG is a hydrogen or functional group.

11. The method according to claim 1 wherein the nanoparticles are derived from self-assembly of amphiphilic block or graft copolymers to form cross-link particles with imaging dye immobilized in the particle via covalent chemical bond in the core of the nanoparticles and alkoxy silane cross-linking resulting in organic/inorganic hybrid materials.

12. The method according to claim 1 wherein said biological cargo-laden nanoparticles include a nanoparticulate imaging probe comprising an oxide core, a biocompatible polymeric shell covalently attached to the oxide core, a dye that produces emissions in response to electromagnetic radiation, a quencher that quenches the emissions of the dye, and a cleavable peptide that covalently binds the probe to a component selected from the group consisting of the dye and the quencher, such that the component is liberated from the probe when the peptide is cleaved, wherein the probe has a size of less than 100 nm and the emission of the dye molecules is quenched when the component is bound to the probe and not quenched when the component is liberated from the probe.

13. The method according to claim 1 wherein said biological cargo-laden nanoparticles include multimodal imaging probes comprising a nanoparticle with one or more imaging components capable of being imaged by one or more imaging modes including luminescence or fluorescent imaging component, X-ray and MRI.

14. The method according to claim 1 wherein said biological cargo-laden nanoparticles are mixed with a vasodilating agent or agents.

15. A device for transdermal delivery of biological cargo to an animal or human, comprising:
biological cargo-laden nanoparticles, said particles including multimodal optical molecular imaging probes, in a form that can be absorbed through the skin, and
a device attachable to said skin for delivering said biological cargo-laden nanoparticle(s) to said skin of an animal or human.

16. The device according to claim 15 wherein said device is attachable to said skin of a human by one of the following:
a patch containing a vasodilating agent or agents,
a patch containing micro needles, or
a patch containing multi-layer time release material.

17. The device according to claim 15, wherein said device is attachable to said skin of an animal by one of the following:
secured to the tail containing a vasodilating agent or agents,
secured to the tail containing micro needles, or
secured to the tail containing multi-layer time release material.

18. The device according to claim 15, wherein said biological cargo-laden nanoparticles comprise any one of the following:
drugs,
vaccines,
biopharmaceuticals,
imaging agents,
multimodal imaging agents,
biomolecules, or
anti-infectives.

19. The device according to claim 15, wherein there are a first plurality of different types of biological cargo-laden nanoparticles located in a corresponding second plurality of separate time release layers.

20. The device according to claim 19, further comprising at least one semipermeable layer located between at least two of said layers in said second plurality to control diffusion rates of said nanoparticles.

21. The device according to claim 15, wherein said biological cargo-laden nanoparticles located in an absorbent section containing a vasodilating agent, said section having a surface for contacting said skin.

22. The device according to claim 21, wherein said surface is adhesive to said skin.

23. The device according to claim 21, further comprising a protective cover surrounding said absorbent section.

24. The device according to claim 23, wherein said protective cover is compressible to clamp the device to said skin.

25. The device according to claim 21, further comprising an opening for receiving a tail of an animal, whereby said tail is contacted by said absorbent section; and means for clamping the device to said tail.

26. The device according to claim 21, wherein said absorbent section is formed in at least one section for surrounding a tail of animal, further comprising means for clamping the device to said tail.

27. The device according to claim 21, further comprising a removable protective layer for said surface.

28. The device according to claim 15, wherein said biological cargo-laden nanoparticles located in an adhesive layer forming a part of said device.
